(19) United States
(12) Patent Application Publication
(10) Pub. No.: US 2016/0045120 A1
(43) Pub. Date: Feb. 18, 2016

(54) SYSTEMS AND METHODS FOR SPIKE SORTING

(71) Applicant: Massachusetts Institute of Technology, Cambridge, MA (US)

(72) Inventors: Alexander Friedman, Brighton, MA (US); Leif G. Garrison, Cambridge, MA (US); Michael D. Keselman, Chestnut Hill, MA (US); Ann M. Graybiel, Lincoln, MA (US)

(21) Appl. No.: 14/460,458

(22) Filed: Aug. 15, 2014

(51) Int. Cl. A61B 5/04; A61B 5/00

(52) U.S. Cl. CPC A61B 5/04001; A61B 5/7264; A61B 5/4064; A61B 5/7221

(57) ABSTRACT

In a system for unsupervised spike sorting, features/dimensions suitable for clustering of the recorded spike signal data are identified, and the feature space is scaled according to the computed respective importances of the various features/dimensions. Clustering is performed on the scaled feature space.

### GOVERNMENT LICENSE RIGHTS

This invention was made with Government support under Grant Nos. MH060379 and NS025529, awarded by the National Institutes of Health, and under Contract No. W911NF-10-1-0059, awarded by the Army Research Office. The Government has certain rights in the invention.

### FIELD OF THE INVENTION

This specification relates generally to automated classification techniques and, in particular, to systems and methods for automated clustering.

### BACKGROUND

In electrophysiology, neural activity is commonly recoded as one or more electrical signals, where the firing or activity of a neuron is represented in the recorded signal as a spike relative to a baseline signal value that usually represents background noise. Subsequent analysis of the recorded signals generally involves spike sorting, i.e., classification of the spikes (also called spike signals) into one or more clusters, where each cluster represents a particular kind of activity of certain neurons. Such classification and/or determination of neural activities often forms the basis for diagnosis of physiological conditions, disorders, etc., and can facilitate beneficial treatment of such conditions and disorders. Fully unsupervised (i.e., automated) spike sorting has been an elusive goal for a number of years. The problem has become more significant with the availability of high channel count, high-density probes that are described below.

Typically, extracellular voltage signals from a local group of neurons are recorded using a probe that has one or more electrodes, where each electrode represents a channel. For example, a probe can be a stereotrode (having two channels) or a tetrode (having four channels). Generally, the electrodes of a probe are arranged to be in close proximity to a single neuron (e.g., within 50 microns from the neuron). As such, a particular neuron's activity can be recorded nearly simultaneously (e.g., with an offset of less than a few, tens, or hundreds of microseconds) on each electrode of a probe. As an example, two tetrodes can be placed 2 mm apart, where each tetrode is a four wire electrode bundle with an internal spacing of 50 μm. A spike from a single neuron is unlikely to appear simultaneously on the two bundles due to the relatively large distance between them, but may be recorded simultaneously on several wires of one particular tetrode.

In spike sorting, there is generally no advantage to considering all eight wires as a single source of intermixed spikes, and the signals from each tetrode (a probe in general) may be sorted separately. If two neurons that generate similarly shaped spikes are equidistant from a single probe and/or electrode, the resulting waveforms recorded at that probe/electrode may be virtually indistinguishable. A larger number of recording sites, i.e., several probes, each having several electrodes (i.e., channels) can minimize the likelihood that two neurons are equidistant from all probes/electrodes. As such, the activities from neurons that generate similar spikes can be distinguished and detected.

Spike sorting generally includes: (1) detection of spike signals or spikes, (2) extraction of distinctive features, e.g., parameters of spike shapes, and (3) clustering of the spikes using the identified features. Spike shape features such as peak-to-peak amplitude, width, and/or principal components are commonly used in spike sorting. In general, however, the features that are best suited or at least well suited for distinguishing one spike cluster from another cannot be determined prior to analyzing the recorded data. Even when the optimal features can be determined, e.g., by analyzing the recorded data, variance in a particular feature's distribution can introduce significant errors in many unsupervised clustering techniques. Manual clustering of the data, as an alternative, is not only highly time-consuming but also is error prone. Manual clustering is virtually infeasible when signals are simultaneously recorded from several channels of several probes. In one approach, spike classes are defined as spike templates. This approach is not reliable, however, when the signal-to-noise ratio (SNR) is high and/or when data are collected simultaneously from a number of channels.

### SUMMARY

In various embodiments of methods and supporting systems described herein, clustering of spike signal data collected from several probes having one or more electrodes can be performed automatically, by a processor, and with high accuracy. This is achieved, in part, by identifying a set of features (also called dimensions) that are determined to be relevant to clustering by analyzing the recorded spike signal data. An importance of each dimension is computed, and the feature space is scaled according to the respective importance of each dimension. The clustering is performed using the scaled dimension space, which can improve the quality of clustering. The quality may be improved further by recursively subclustering each cluster, and by selecting different dimensions and/or by re-computing the dimensional importances in each recursion.

Accordingly, in one aspect, a computer implemented method for spike sorting includes: (a) receiving, in memory, data representing a number of spike signals; (b) identifying a set of dimensions associated with the several spike signals; and (c) selecting a first subset of dimensions from the set of dimensions. Each dimension in the set corresponds to a feature of the spike signals. The method also includes: (d) for each dimension in the first subset, computing by a processor a corresponding dimensional importance; and (e) for each dimension in the first subset, scaling by the processor the corresponding feature in the spike signals according to the corresponding dimensional importance. The method further includes: (f) clustering the spike signals having the scaled features to obtain an initial set of clusters.

The set of dimensions may include one or more of: a peak voltage corresponding to a channel for the spike signals, a principal component (PC) of a channel for the spike signals, and a peak PC of a channel for the spike signals. Computing the corresponding dimensional importance for a dimension can include projecting, by the processor, the spike signals along the dimension. The processor may identify via iterative fuzzy c-means clustering of the projected spike signals, a clustering that maximizes a clustering score, and may designate a dimensional importance to the dimension, based on the maximized clustering score. Designating the dimensional importance may include setting the dimensional importance to zero if the maximized clustering score is less than a selected threshold, and otherwise setting the dimensional importance to a metric that results from a function of the number of clusters in the clustering that maximizes the clustering score. In some embodiments, the selected threshold is 0.75, and the metric is the square of a value that is the number of clusters minus one.

In some embodiments, the method includes identifying a first dimension and a second dimension, wherein a first clustering corresponding to the first dimension is similar to a second clustering corresponding to the second dimension. The similarity may be determined according to a similarity metric. The method may also include comparing the respective dimensional importances of the first and second dimensions, and resetting the dimensional importance of the dimension having a lower dimensional importance to zero. Clustering the spike signals having the scaled features may include iterative fuzzy c-means clustering.

In some embodiments, the method includes: (g) for each cluster in the initial set of clusters: selecting a second subset of dimensions from the set of dimensions, such that at least one dimension in the second subset is different from any dimension in the first subset. In this embodiment, the method further includes: for each dimension in the second subset, computing by the processor a corresponding dimensional importance. For each dimension in the second subset, the processor may scale the corresponding feature in the spike signals in the cluster according to the corresponding dimensional importance, and clustering the spike signals having the scaled features in the cluster to obtain a subset of clusters within the cluster. In this particular embodiment, the method also includes: (h) designating all subsets of clusters resulting from step (g) as a final set of clusters.

Each cluster in the final set of clusters may be refined iteratively. Iteratively refining a cluster may include determining a cluster core, and scaling each dimension in a plurality of dimensions associated with the cluster according to a distance, in the respective dimension, between the cluster core and spike signals in the cluster that are not within the cluster core.

In some embodiments, the method includes filtering out a portion of the received data representing the spike signals, using a first signal-to-noise ratio (SNR) threshold. The filtered out portion of the received data may be re-filtered using a second SNR threshold that is lower than the first SNR threshold, and the method may include repeating steps (b) through (h) using the re-filtered data representing the plurality of spike signals. The filtering may include computing an SNR for each spike signal in the plurality of spike signals, and removing, from the data representing the spike signals, data corresponding to spike signals having an SNR less than the first SNR threshold. In some embodiments, the filtering includes, additionally or in the alternative, spatially partitioning the spike signals into a number of bins, and computing a respective bin density for each bin. At least one spike signal may be removed from the data representing the spike signals, where each removed spike signal had been partitioned into a bin having a density less than a threshold value. The threshold value can be obtained from a function of bin densities of neighboring bins.

In some embodiments, the method includes, prior to step (b), removing, from the data representing the plurality of spike signals, all but one spike signals that are near-simultaneous. After iteratively refining each cluster, each of the removed spike signals may be added to a cluster according to a selected dimension, e.g., a peak principal component value, of the respective removed signal. In some embodiments, the method includes computing at least one quality metric for each cluster in the final set of clusters. A qualitative value, e.g., good, medium, or bad, may be designated to each cluster based on one or more corresponding quality metrics. Computing the one or more quality metrics may include computing an Lratio for the cluster, a tightness of the cluster, an incompleteness of the cluster, a percentage of inter-spike intervals that are shorter than a specified threshold, and/or an isolation distance.

In some embodiments, the spike signals are obtained from several channels, and the method further includes aligning spike signals from each of the several channels by aligning spike signal peaks across the several channels. The method may include removing spike signals in a selected time period if a number of spike signals in the selected time period exceeds a specified threshold. In some embodiments, the method includes determining, for at least one dimension of the clusters, a separability of each cluster in that dimension, and dissolving one or more clusters that are determined to be separable. The clusters can be selected from the initial set of clusters and/or the final set of clusters, optionally after refinement thereof, as described above.

In another aspect, a computer system includes a first processor and a first memory coupled to the first processor. The first memory includes instructions which, when executed by a processing unit that includes the first processor and/or a second processor, program the processing unit to: (a) receive, in a memory unit including the first memory and/or a second memory, data representing a number of spike signals; (b) identify a set of dimensions associated with the several spike signals; and (c) select a first subset of dimensions from the set of dimensions. Each dimension in the set corresponds to a feature of the spike signals. The instructions also program the processing unit to: (d) for each dimension in the first subset, compute a corresponding dimensional importance; and (e) for each dimension in the first subset, scale the corresponding feature in the spike signals according to the corresponding dimensional importance. The instructions further program the processing unit to: (f) cluster the spike signals having the scaled features to obtain an initial set of clusters. In some embodiments, the second memory coupled to the second processor can receive through a network the instruction stored in the first memory.

In another aspect, an article of manufacture that includes a non-transitory storage medium has stored therein instructions which, when executed by a processor program the processor to: (a) receive, in memory coupled to the processor, data representing a number of spike signals; (b) identify a set of dimensions associated with the several spike signals; and (c) select a first subset of dimensions from the set of dimensions. Each dimension in the set corresponds to a feature of the spike signals. The instructions also program the processing unit to: (d) for each dimension in the first subset, compute a corresponding dimensional importance; and (e) for each dimension in the first subset, scale the corresponding feature in the spike signals according to the corresponding dimensional importance. The instructions further program the processing unit to: (f) cluster the spike signals having the scaled features to obtain an initial set of clusters.

Various implementations of each of these aspects include corresponding systems, apparatus, and/or computer programs.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 depicts an example of a process of spike sorting, according to one embodiment;

FIG. 2A depicts waveforms corresponding to four channels of a spike signal, according to one embodiment;

FIG. 2B depicts scaling of a dimension space, according to one embodiment;

FIG. 2C depicts initial clustering, according to one embodiment;

FIG. 2D depicts final clustering prior to refinement, according to one embodiment;

FIG. 2E depicts refined clusters, according to one embodiment;

FIG. 2F depicts refined clusters after a number of iterations at different SNR filtering thresholds, according to one embodiment; and

FIGS. 3A and 3B depict a system, according to one embodiment, that is configured to perform various spike sorting operations described herein.

### DETAILED DESCRIPTION

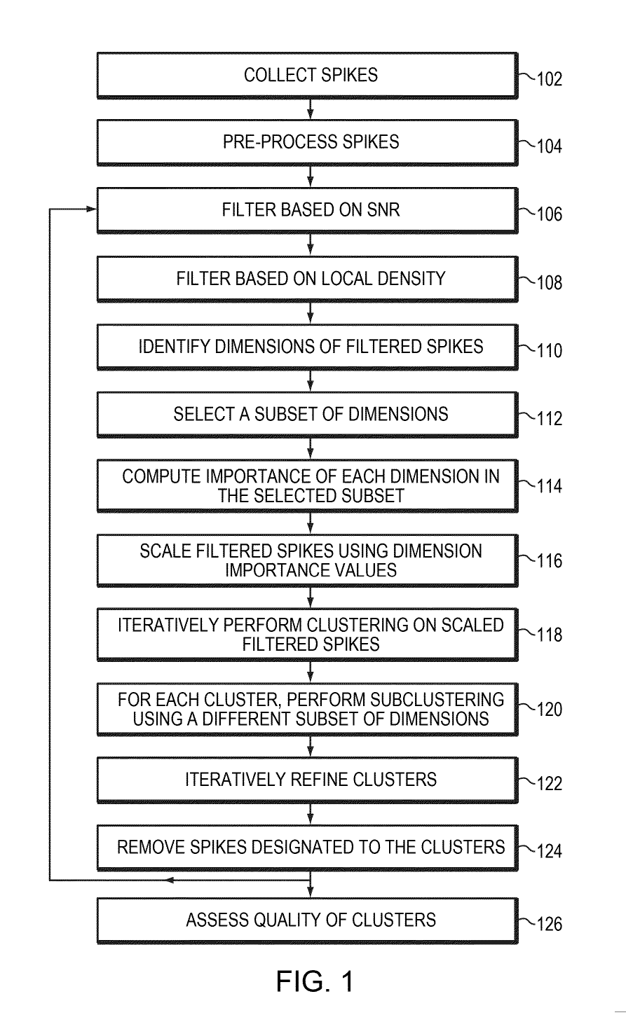

In a process 100 depicted in FIG. 1, spike signals are collected in step 102. These signals can be collected from one or more probes, where any probe can collect data via one or more electrodes/channels. A spike signal that is indicative of the activity of a single neuron is generally recorded as a set of several waveforms, where each waveform recorded via a different channel/electrode of a probe. During data acquisition in the step 102, a recording window (typically a 1 ms window) is triggered by a threshold crossing, e.g., the recorded voltage exceed a specified threshold such as 15 mV, 10 mV, etc., in a low noise environment, or 30 mV, 50 mV, 100 mV, etc., in a relatively high noise environment. The voltages recorded during a particular window can represent one or more spike signals. If a single electrode/channel records two spikes from two different neurons simultaneously, the recorded signal would be a combination of individual spike signals, and the signal from that channel is generally not useful in subsequent analysis. Other channels and/or probes, however, may be able to discriminate the two spikes.

In step 104, the collected spikes are pre-processed. Pre-processing generally includes initial selection of the spike signals data (also referred to as spike signals) and enhancement of the signal-to-noise ratio (SNR) of the selected spike signals. In some instances during data acquisition, when a recording window is triggered, additional nearly simultaneous spikes may also be recorded within the same window. As described herein, “nearly simultaneous” generally means spike signals that overlap each other but are offset by a short duration of time such as less than 1 ms. Thus, a “nearly simultaneous” spike can be an additional spike that is recorded within the same recording time window after the recording is triggered by a previous spike. In some embodiments, only one of the nearly simultaneous spike signals is selected for further processing and the remaining nearly simultaneous spike signals are temporarily removed from the data to be processed. After clustering is completed, these nearly simultaneous spike signals are added to an appropriate cluster, as described below. For example, the spike signal that triggered the recording window can be selected. In some embodiments, if a recording window contains nearly simultaneous spike signals, all of them can be temporarily removed. In general, the shape and principal components of a nearly simultaneous spike are likely altered by being separated from one or more other spike signals recorded in the corresponding window, and potentially by the voltage changes of such other spikes. Therefore, in some embodiments, the nearly simultaneous spike signals may not be used in clustering. The peak voltage is generally not significantly affected (e.g., less than a 1% or 10% change relative to the peak voltage that would be observed if only one spike signal were recorded in that window), by other spikes recorded in the same window, however.

The spike signals recorded via two or more channels of a probe in a particular recording window typically represent an activity in a single neuron. In some embodiments, these signals are therefore aligned in the step 104, for example, to prevent the generation of misleading results in subsequent principal component analysis (PCA) and/or in the computation of the “tightness” measure discussed below. The spike signal alignment can be achieved by shifting the signal waveforms from the different channels horizontally (i.e., along the time axis) so as to align their respective peaks. Prior to the shifting of the waveforms, the approximately 1 ms recording window may be expanded, e.g., to about a 1.25 ms window, so that substantially all of the spike signal recorded in that window is preserved when the waveform is shifted for alignment thereof. As used herein, substantially generally means at least 50%, at least 80%, at least 95%, etc., of the waveform.

In one embodiment, the spike waveforms are first interpolated to prepare them for alignment. Data recorded using modern equipment and some older systems generally has the resolution sufficient for linear interpolation. A 32-point recorded waveform is converted into a 120-point waveform via interpolation. For each channel, the 1 ms recording window is expanded to 1.25 ms, such that 75% of the additional 0.25 ms is added to the beginning, and the remainder is added to the end. This expansion permits shifting waveforms horizontally during alignment without excessively truncating them. In this embodiment, the peaks are aligned to the 20% mark in the original 1 ms recording window, which is about the 35% mark in the expanded alignment window. The shifted waveforms are padded with zeros and/or are truncated at the edge of the 1.25 ms window. It should be understood that the specific values such as 1.25 ms expansion window, the number of points before and after interpolation, and 75%-25% division of the additional window are illustrative only.

In some embodiments, a stationarity filter is applied in the step 104. In some situations, a loose connection between a probe and the surface from which signals are to be recorded and/or malfunction of the probe components introduce noise that can be in the form of rapid voltage fluctuations that are similar to the spike signals representing the neural activity to be recorded. Typically, however, the noise fluctuations occur more rapidly than the spike signals. Therefore, in some embodiments, the spike signals are distributed across time bins (e.g., bins of durations such as 10 ms, 50 ms, 100 ms, 125 ms, 200 ms, etc. The signals from any bin that includes a significantly large number of signals or fluctuations are considered to be noise signals and are removed from the data representing the spike signals. A significantly large number can be a number that is greater than the mean number of signals in the various bins by a multiple (e.g., 3, 5, etc.) of a standard deviation of the number of signals in those bins. Typically, a broad range of peak voltages are recorded on all functional channels, and the recorded peak voltages recorded tend to be linearly related to each other.

In step 106, the spike signal data are filtered using a signal-to-noise ratio (SNR) filter, to select the spike signals that have an SNR greater than a selected threshold SNR. To compute the SNR for a spike signal, a spike signal amplitude is computed as a difference between a peak value of the signal waveform (e.g., peak voltage) and a valley value of the signal waveform. In a spike signal, the peaks and valleys are the maximum and minimum signal values (e.g., maximum and minimum voltage values, in a voltage signal), respectively. If waveforms corresponding to a single observed entity (e.g., a single neuron) are obtained via more than one channels, a respective waveform amplitude is computed for each of those waveforms. These waveform amplitudes, though associated with a single spike signal, can be different. Typically, the differences in the amplitude across different channels result from differences in impendence and proximity of the sensor/wire to the signal source (e.g., a neuron).

To normalize the amplitudes for differences among the various channels the initial z-score of the amplitude for each channel is computed individually, where the initial z-score can be the multiple in terms of the standard deviation of a baseline mean signal level for that channel. In some embodiments, the z-score is the number of standard deviations each spike's amplitude is above the mean spike amplitude on that channel. For each spike signal waveform, the best channel, i.e., the channel having the maximum initial z-score relative to the other channels is selected. Thereafter, the amplitude values corresponding to the channel selected for each recording window are normalized by computing final z-scores of those values. The mean value used in the final z-score computation is the mean of the amplitudes of the waveforms from the selected channels. For each spike signal corresponding to a particular recording window, the final z-score for the amplitude of the waveform from the channel selected for that window is designated as the spike signal SNR. Each spike signal, represented by a number of waveforms, is thus assigned a single SNR value.

In a particular iteration of some of the steps (e.g., steps 112-120) of the process 100, a spike signal is processed only if that spike signal were not processed in a previous iteration and the SNR thereof is greater than the SNR threshold level selected for that particular iteration. In each subsequent iteration of the steps of the process 100 the SNR threshold level is decreased, as described below. In the final iteration, the filter may not be applied, i.e., processing may be performed on all remaining spike signals that were not processed in any of the earlier iterations because their respective SNRs were below the SNR threshold applied in the penultimate iteration. As an example, the SNR threshold levels can begin at 2 and decreases to 1.5, and 1.0, in sequence, and be zero for the final iteration.

In some embodiments, local density based filtering is applied in step 108, generally to improve performance of the clustering steps. In order to enhance the separability of clusters, the local density based filter can remove background spikes that are not part of any cluster. To this end, a multi-dimensional peak space is spatially partitioned into bins. In one embodiment, the four dimensions correspond to the peak voltages recorded on the four channels. Techniques such as spatial binning, where the bin dimensions are proportional to the standard deviation of peak voltage in each channel, can be used for the spatial portioning. If the density of a particular bin, i.e., a number of spike signals partitioned into that particular bin, is less than a value computed as a function of the respective densities of the neighboring bins, the spike signals partitioned into that particular bin are removed from further processing at steps 110-120 of the process 100. This can facilitate the removal of spike signals near edges of clusters while clustering is performed, because it may be unclear whether one or more of the removed spikes belong to a particular cluster or the neighbor thereof. The spike signals omitted from the processing at the steps 110-120 can be analyzed subsequently in the refinement step 122, and can thus be designated to a suitable cluster.

After the SNR-based filtering in step 106 and/or local density filtering in step 108, one or more of the core clustering steps are performed. The core steps include three main parts: (1) initial feature space construction, dimension evaluation, and spatial transformation, in which a transformed feature space is constructed by scaling dimensions (features) according to their importance, (2) clustering and subclustering, and (3) refinement. The stationarity filtering, SNR filtering, and/or local density based filtering, and one or more of the core clustering steps may then be iterated, in order to find additional clusters that were not found in a preceding iteration. After each iteration, the clusters derived in that iteration are removed from the spike signal data to be processed in a subsequent iteration. As described above in the discussion of the step 106, in each iteration, the SNR threshold level is decreased. In the final iteration, SNR filtering (step 106) and local density based filtering (step 108) may be omitted or, equivalently, the respective applicable filter thresholds are set to zero, in order to derive valid clusters containing spike signals that may have been filtered out, and thus not processed, in earlier iterations.

In step 110 a number of dimensions of the spike signal data (which may be preprocessed and/or filtered as described above) are selected. In general, a dimension represents a feature of the spike signals. Recall that each spike signal is represented by one or more waveforms recorded via one or more channels. As such, typical features include peak values (e.g., voltages) of the different waveforms corresponding to different channels, one or more principal components (PC), e.g., PC1, PC2, etc. of one or more of those waveforms, obtained via principal component analysis (PCA), peak PC values, etc.

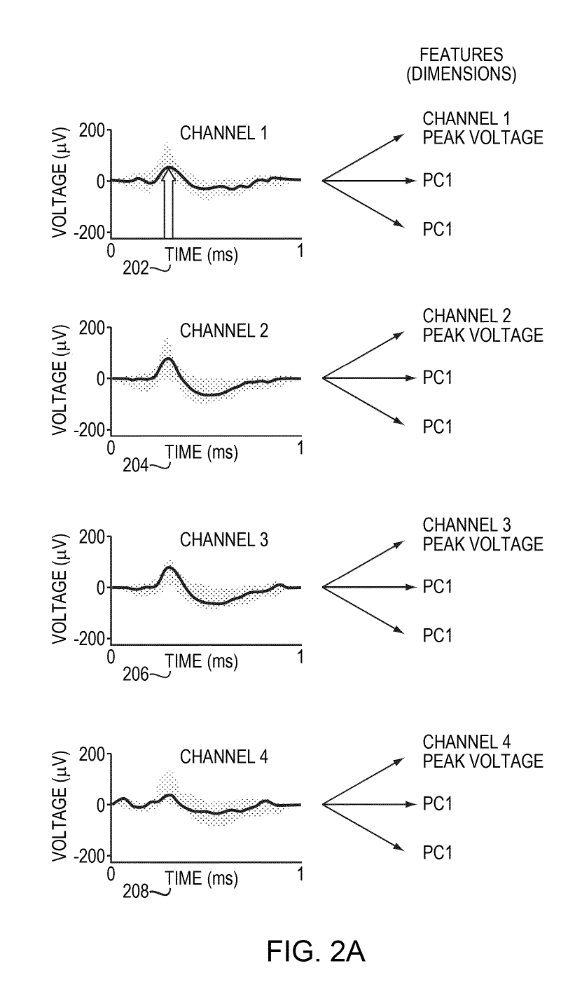

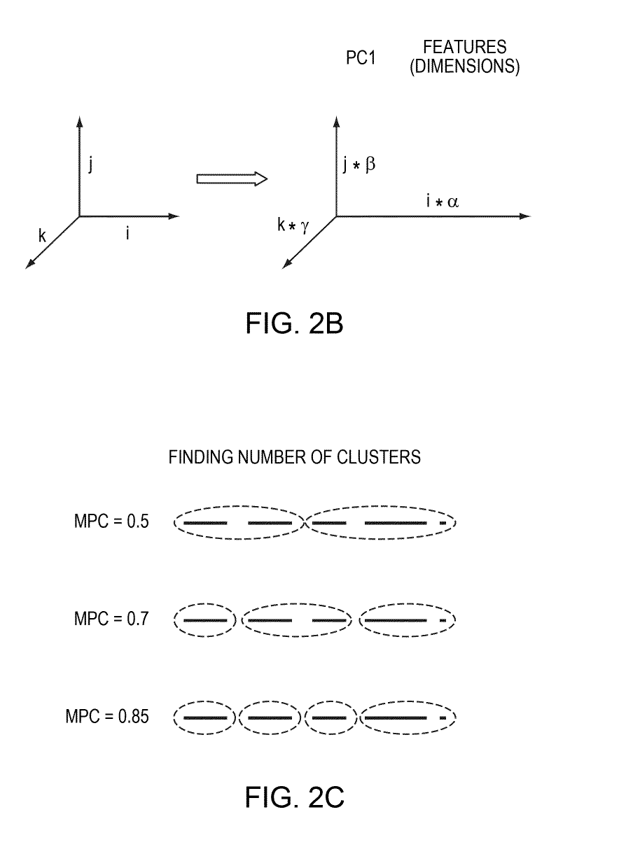

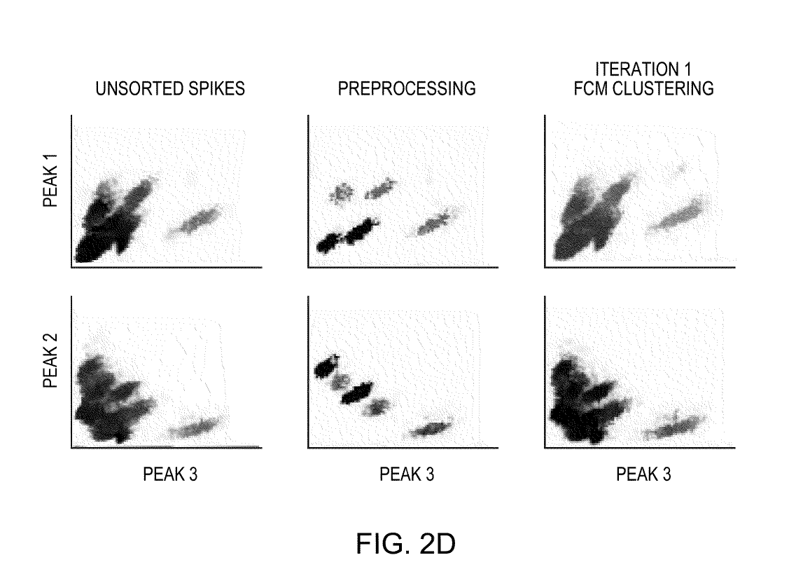

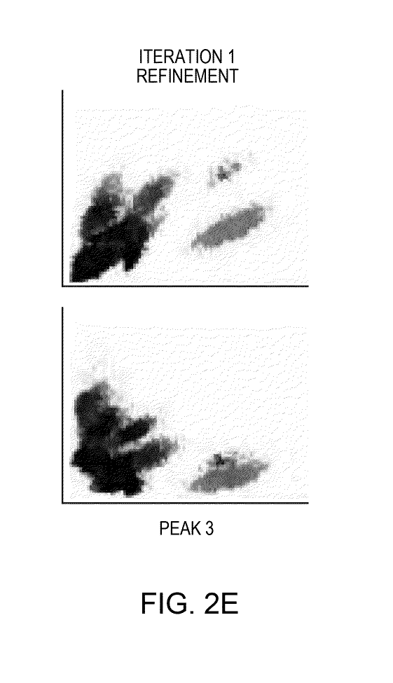

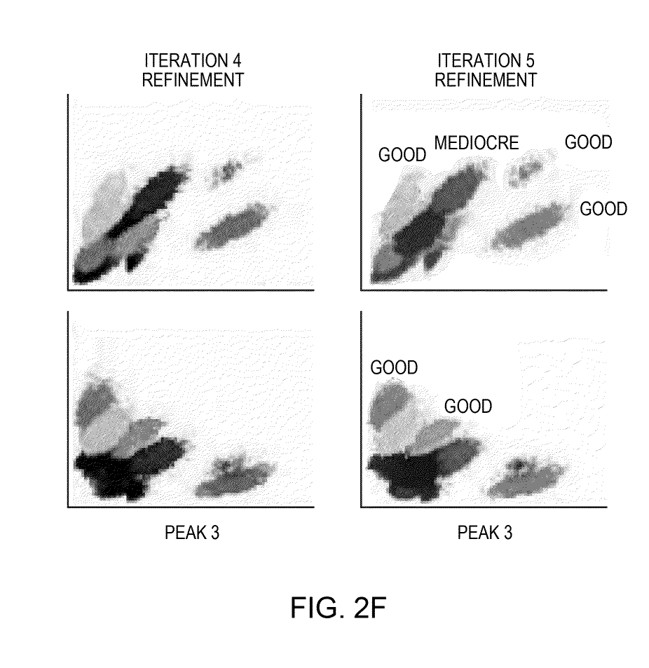

As depicted in FIG. 2, a spike signal includes waveforms 202-208 recorded via four tetrode channels. The feature space of the signal includes 15 dimensions, namely, 4 peak voltage dimensions (one for each tetrode channel), 8 PC dimensions (PC1 and PC2 of each waveform), and 3 peak PC dimensions (PPC1, PPC2, and PPC3). In calculating the peak PC dimensions, the initial four dimensions are the peak voltages recorded on the four channels. In contrast, while calculating the regular PC, the initial n dimensions (n≧1) are the voltages at the n time points of the interpolated, peak-aligned, and padded waveform from one channel. The peak voltages on the four channels together can be viewed as a signature reflecting, in part, the location of the neuron relative to the four wires of the tetrode. It should be understood that the number of channels, the number of dimensions, and the particular dimensions described above are illustrative only. In general fewer or more than four channels (e.g., 2, 6, 10 channels) and/or fewer or more than 15 dimensions (e.g., 5, 8, 20, 26, 35, etc.) dimensions can be identified for subsequent analysis. Different dimensions, such as peak voltages of some but not all channels, PC1 or PC2 of some but not all channels, other principal components and various peak PC components of some and/or all channels can be identified as well, for subsequent analysis.

In some embodiments, while performing the PCA for a particular channel, a mean subtraction is applied to the waveform in that channel in such a manner that the PCA is focused on the variability in the shape of the waveform rather than on vertical shifts of the whole waveform. Specifically, the initial feature space includes several dimensions where a particular dimension is the voltage at one time point of the waveform (which can be interpolated, peak-aligned, and/or padded within an expanded time window) corresponding to one of the several channels. From the voltage values of a particular waveform, the mean voltage of that particular waveform may be subtracted instead of subtracting from the waveform the mean of all waveforms, computed over time, in that particular channel. This can cause the PCA to be focused on the variability due to a background of multiunit (i.e., multi-neuron) activity, the variability that is discovered to be relevant to clustering. In contrast, while computing peak PCs, from the waveform in a particular channel the mean of all waveforms, computed over time, in that particular channel is subtracted. Each dimension (peak voltage, PC, or peak PC) may independently z-score normalized, using a distribution (e.g., mean and standard deviation) of the peak voltage, PC, and/or peak PC over all spikes (i.e., over time).

Once the initial feature space is constructed, i.e., an set of dimensions is selected in the step 110, a subset of dimensions is selected in the step 112. For each of the selected dimensions from the subset, a respective dimensional importance, used in weighting the dimensions via the spatial transformation described below, is calculated in step 114. The importance of a dimension is based on the number of valid clusters in the projection of the distribution of the spike data onto a one-dimensional subspace corresponding to that dimension. The data points in the distribution are projected from an n-dimensional space (corresponding to n features) onto a one-dimensional subspace. Thus the data points are represented as points in a one-dimensional distribution. In one embodiment, the evaluation of clusters is performed by a combination of fuzzy c-means (FCM) clustering, to find the clusters, and the computation of the modified partition coefficient (MPC) corresponding to that particular clustering, to assess cluster validity.

The FCM clustering requires the number of clusters, c, as one of the inputs to the clustering process. To find the number of clusters that optimizes the cluster validity, the step 114 iteratively performs FCM clustering on the projection of the distribution of the spike data in a selected dimension by varying c in each iteration. Each time an FCM clustering is performed, the MPC value m for that iteration is calculated. The MPC ranges between 0 and 1, and the maximum value of m, mₘₐₓ from the several iterations, and the corresponding number of clusters cₘₐₓ, are used in dimension evaluation. In one embodiment, if mₘₐₓ<0.75, the partitioning (i.e., clustering) resulting from the FCM clustering in that dimension is determined to have described the spike signal data poorly. As such, the dimensional importance for that dimension is set to zero, and the dimension is removed from multi-dimensional clustering performed subsequently. Otherwise, the dimensional importance is set to (cₘₐₓ−1)², thereby giving dimensions generating more identifiable clusters than other dimensions more weight. Other functions of cₘₐₓ, e.g., the number cₘₐₓ itself, can also be used. Clustering methods other than FCM clustering can also be used.

The MPC, an index of cluster validity, is a specific modification of the partition coefficient that was discovered to reduce the monotonic tendency of the partition coefficient with respect to the number of clusters c. The MPC value m is defined as:

$$m = 1 - \frac{c}{c - 1} ( 1 - {V}_{PC} )$$

where V_{PC} is the partition coefficient given by:

$${V}_{PC} = \frac{1}{n} {\sum }_{i = 1}^{c} {\sum }_{j = 1}^{n} {u}_{ij}^{2}$$

Here, n is the number of vectors (e.g. spikes) in the multidimensional space, c is the number of clusters, and uᵢⱼ refers to the entries in the partition matrix U. In one embodiment, uᵢⱼ is the degree of membership of spike j in cluster i on a scale from 0 to 1.

After computing the dimensional importance for all of the dimensions in the selected subset of dimensions, the spike signal data are spatially transformed, i.e., scaled, in step 116 using the respective dimensional importance values. In preparation for the FCM clustering of the multidimensional distribution of the spike data, a transformed feature space is constructed by weighting (scaling) each dimension by its dimensional importance as determined in the dimension evaluation step, as depicted in FIG. 2B. In some embodiments, prior to this transformation, the projection of the distribution onto each dimension is normalized independently using a z-score. To compute the z-score, the mean and standard deviation of each dimension in the multi-dimensional space (e.g., the peak voltage, PC, and/or peak PC) over all spikes (i.e., over time) is computed. From the dimensional value of each spike signal along each dimension, the computed standard deviation corresponding to that dimension is subtracted, and the result is divided by the corresponding mean. This normalization can adjust for differences between properties of the channels, such as differences in channel impedances.

Having prepared a suitable scaled space, in step 118 a clustering (e.g., the FCM clustering) is performed several times on the distribution in this multi-dimensional scaled space, assuming a different numbers of clusters c in each iteration. The number of clusters that maximizes the MPC value, and the corresponding clustering (as depicted in FIG. 2C) are selected as an initial set of clusters. In some embodiments, instead of randomly initializing the FCM, a heuristic that deterministically produces the initial centers of each cluster is employed.

Specifically, in one embodiment, the computation first assumes a single cluster and assigns the centroid of the data as the initial cluster centroid position. When the corresponding iteration of the FCM computation is completed, a new centroid position is obtained for the single cluster. The next iteration then assumes two clusters and assigns the centroid position found in the first iteration and a second centroid adjacent to the first centroid as the two initial cluster centroid positions of the two clusters, respectively. When the second iteration of FCM is completed, a pair of centroid locations are determined. The iterations may continue, increasing the number of clusters in each iteration, and designating the centroids obtained in the immediately preceding iteration as the initial centroids of all but one clusters in the present iteration. In one embodiment, the initial centroid of the newly added cluster is defined as the centroid of the other initial centroids. This heuristic can make FCM computation deterministic, allowing for obtaining consistent results. The heuristic may not guarantee a global minimum for the FCM objective function.

Following the first round of clustering, clusters within clusters are identified in step 120 by applying the same steps used in the initial clustering. In particular, the PCA may be applied only to the spikes within a particular cluster, so as to form a second subset of dimensions that includes at least one dimension not included in the first subset of dimensions. The dimensions in the second subset are selected and evaluated in a similar manner as step 114. If one or more dimensions are determined to meet the separation criteria, i.e., the respective dimensions generate two or more clusters, and have been designated a respective nonzero dimensional importance, the same clustering process as that in the step 118 is applied to the spikes data within the particular cluster. In some embodiments, the subclustering is applied recursively until there are no dimensions that meet the separation criteria. These steps are performed for each cluster included in the initial cluster set, so as to obtain a final set of clusters.

The final set of clusters obtained in the step 120 can be refined in step 122. The clusters in the final set of clusters may not have the tightest boundaries (as depicted in FIG. 2D), for example, due to the removal of spikes during filtering and the inherent limitations of the clustering methods employed in the steps 118, 120, such as FCM clustering. Therefore, in the optional step 122, each cluster in the final set of clusters is reduced to a core and a new boundary therefor is determined. Specifically, each cluster is reduced to a core that normally contains about 30% of the cluster's spikes that are closest to the centroid of the cluster. If the number of spikes in a cluster is less than a specified threshold, about 60% of the spikes may be included in the core. The centroid can be the arithmetic mean of all the points in the cluster. Thereafter, in each dimension, a valley in the distribution of Mahalanobis distances is identified and spikes that are closer to the cluster core than the valley are included in the cluster.

Along each dimension, the Mahalanobis distance between the rest of the spikes and the spikes in the cluster core is computed. The dimension is scaled using the computed distance, e.g., using a scaling factor of one minus the computed Mahalanobis distance. The dimensions are scaled so as to emphasize dimensions with greater separation between the cluster and the rest of the data. The cluster core is expanded to obtain a refined cluster using the scaled dimensions and the Mahalanobis distance.

The above process of reducing the cluster to a core and the re-growing the core using the Mahalanobis distance distributions along the various dimensions of the cluster is repeated, e.g., for a specified maximum number of iterations, or until it is observed that the core does not change significantly in the next iteration. In some embodiments, other distance measures, such as Euclidean distance, can be used an alternative to Mahalanobis distance.

The clusters obtained after refinement generally represent an accurate clustering, such as the example of clusters shown in FIG. 2E. For the sake of illustration and clarity, two two-dimensional projections, Peak_1 vs Peak_3 and Peak_2 vs Peak_3, respectively, of the multi-dimensional space are depicted. In general, however, clusters in the multi-dimensional space are refined. Recall, these clusters were generated for a particular SNR threshold that was selected in the step 106. After the spike signal data corresponding to these clusters is removed from further processing in step 124, the spike signals that have not been processed yet still remain. These spikes were filtered out by the particular SNR threshold used in the step 106.

In order to cluster at least some of these previously unprocessed spike signal data, the step 106 is repeated and a new SNR threshold that is lower than the previous one is selected. The spike signal data now selected using the new lower threshold are processed by repeating one or more of the steps 108-124, so as to find new clusters or to designate additional spike signals to the clusters found in any of the previous iterations of the steps 106-124. The number of iterations can be selected and, in the last iteration, in the pre-processing step 104, the stationarity filtering may be applied. The SNR based filtering in the step 106 and the spatial filtering in the step 108 may be omitted, however, so as to process all remaining spike data not processed in the previous iterations. An example of refined clusters obtained after five iterations in which the SNR filtering threshold is successively lowered is shown in FIG. 2F.

As part of cluster refinement in the step 122, or before the removal of the spike data designated to clusters from processing in subsequent iterations in step 124, “bad,” i.e., non-unimodal clusters may be dissolved. Additionally or in the alternative, some clusters may be merged and/or overlap between clusters may be reduced or eliminated. In general, bad clusters that are not unimodal can be dissolved at any step when the clusters should be unimodal. As such, in various embodiments, if a cluster is not unimodal when it should be, the spikes designated to that cluster are considered unsorted, and may be sorted, as described above, in a subsequent iteration. A bad cluster found at the end of the last iteration can be removed permanently.

To dissolve bad clusters, after any iteration is completed, each dimension may be tested individually. In particular, a cluster is dissolved, i.e., the spike signals designated to that cluster are considered to be unsorted, if it is evaluated as potentially separable along any dimension according to the following test. Let v be any local minimum in the projection of the distribution of the spike signal data onto a particular dimension. Let p be the largest local maximum on either side of v. The maxima can represent centers of potential subclusters, and the minima can represent the dip between the potential subclusters. If v<¾ p for any v, the spikes data corresponding to that particular cluster are potentially separable along the dimension for which the above relation holds. The cluster dissolved at this stage and the spikes designated thereto are typically re-clustered in a subsequent iteration. If any two clusters have a high overlap, i.e., a significant fraction of spikes (e.g., 60%, 65%, 85% or more, etc.) belong to both clusters, the two clusters can merged into a single cluster. If two clusters have a low but nonzero overlap (e.g., 5%, 10%, less than 40% clusters belong to both clusters), the overlapping spikes are assigned to one of the two clusters by minimizing respective Mahalanobis distances between each spike signal in the overlapping region and each overlapping cluster. The Mahalanobis distance is computed for a distribution of the spike signals data designated to a respective overlapping cluster.

After all spike signal data have been clustered, post processing is performed and quality of the clusters is determined in step 126. Recall, the spike signal waveforms of different neurons recorded by a single probe within a single 1 ms window were temporarily removed in step 104. These waveforms may be separated from each other using appropriate timestamps. The separated waveforms may then be assigned to different clusters based on a dimension or feature that is relatively less affected due to the presence of other signals in the recording window than other dimensions/features. Peak voltages are an example of a feature that can be used for such classification.

Cluster quality can be computed using one or more of several criteria. For example, an Lratio is a measure of how close a cluster is to the rest of the data based on Mahalanobis distance Another measure of closeness of the two distributions (clusters) to each other, the Bhattacharyya distance, can also be used. In general, the smaller the Lratio measure the better the quality of clustering according to this measure. Tightness is a measure of spike waveform consistency represented as

$$\frac{max ( peaks )}{10 \times std ( peaks )} ,$$

where “peaks” is a vector of all peak voltages for the channel that has the highest peak voltage. Generally, a larger tightness value indicates a better clustering according to the tightness measure. Tightness computed differently, as the discrepancy between PC1 of each waveform in a cluster and PC1 of a template waveform (e.g., the mean waveform), can be used to test that an identified cluster is not, in fact, an artifact.

Incompleteness is another measure that estimates a fraction of the cluster truncated by the spike recording threshold, assuming that the cluster has a normal distribution. To compute the incompleteness, the cluster is assumed to be symmetric and not normal. The amount of the theoretical cluster that is below a threshold, assuming it is centered at its observed mean, is computed using data corresponding to the best channel, determined as described above. Generally, a smaller incompleteness value indicates a better clustering according to this measure. The measure short ISIs, represents a percentage of interspike intervals shorter than a specified threshold (e.g., 3 ms refractory period), where, typically, the smaller the short ISIs, the better the clustering according to this measure. The isolation distance is another measure, where a larger value of this measure generally indicates a relatively better clustering.

In some embodiments, a summary of cluster quality may be provided qualitatively as “good,” “medium,” or “bad” for each cluster based on a combination of Lratio, tightness, incompleteness, and/or the number of spikes in the cluster. For example, a cluster can be designated as good if Lratio<0.1, tightness>0.8, incompleteness=0, and number of spikes in the cluster>800. The cluster may be designated as medium if Lratio<0.3, tightness>0.5, incompleteness<0.1, and number of spikes>I 000. The cluster may be designated as bad if Lratio<0.5 and tightness>0.5, incompleteness<0.5, and number of spikes>1000. It should be understood that the specific values used above are illustrative only and that the thresholds can be set in a different manner, according to whether a high or low value of a particular measure indicates relatively better clustering, as described above.

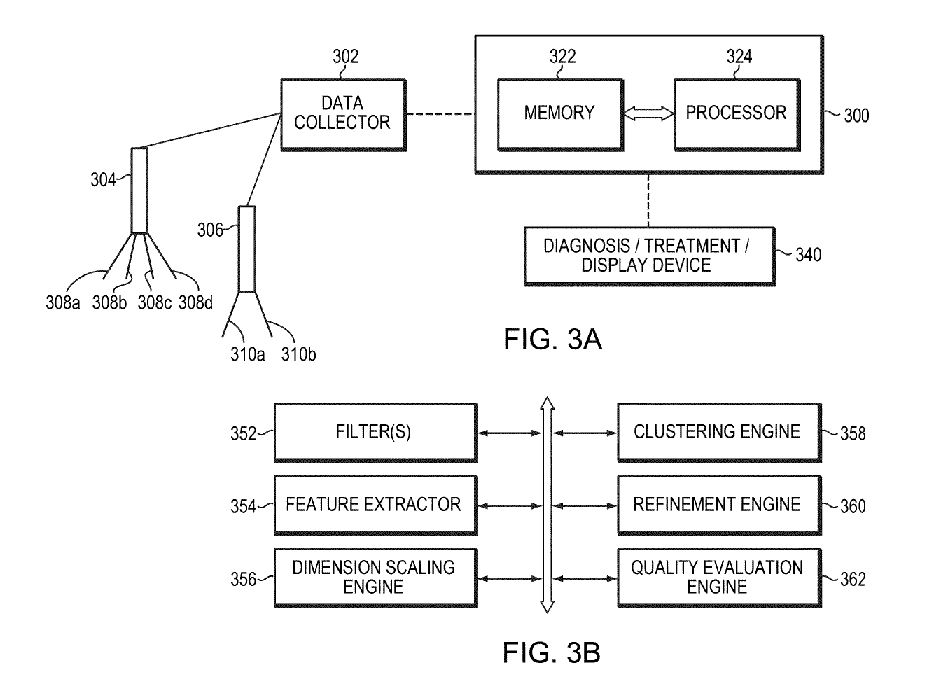

With reference to FIG. 3A, a system 300 can receive and process spike signal data received from a data collector 302. The data collector 302 records spike signals via one or more probes, e.g., probes 304, 306. In general, each probe can have one or more electrodes, corresponding to one or more channels per probe. The probe 304, for example, has four electrodes 308a-308c and the probe 306 has two electrodes 310a, 310b. The recorded data are received in memory 322 coupled to a processor 324. The processor 324 is programmed to perform various spike sorting operations described above, such as the operations described with reference to FIG. 1. The processor 324 can store and access partial results in the memory 322. Examples of the partial results include dimension importances computed by the processor, spike signals data scaled according to the dimension importances, initial and final cluster sets; cluster cores computed during refinement, refined clusters, and various quantitative and qualitative metrics associated with the refined clustering. The system 300 can provide the partial and/or final results to one or more diagnosis, treatment, and/or display devices 330.

With reference to FIG. 3B, the processor 324 can be programmed as one or more modules that can operate sequentially and/or in parallel, communicating with each other and/or with the memory 322. In some embodiments, the processor 324 may be configured as all of the modules, while in other embodiments the processor 324 may be configured as one or more modules, and the other module or modules may be implemented by another processor, co-processor, or a hardware unit. Typical modules include one or more filters 352, which can implement the stationarity, SNR based, and/or spatial filtering described above. The feature extractor 354 can identify the dimensions relevant to clustering, and the dimension scaling engine 356 can compute the dimension importances and scale the spike signal data according to the computed importances. The clustering engine 358 can iteratively and/or recursively perform clustering, and the refinement engine 360 can refine the clusters in the final set of clusters. The quality evaluation engine 362 can compute one or more of the metrics described above, such as Lratio, tightness, incompleteness, short ISIs, etc., and the qualitative metrics described above. It should be understood that the modules 352-362 are illustrative only and that the spike sorting operations can be implemented by as few as one and more than six modules. In different embodiments, any module can be configured to perform any one spike sorting operation or combinations of operations that are different than the operations or combinations of operations that are designated to the modules 352-362.

Clustered neural data can be used for diagnosis of various conditions, brain disorders, etc., and in global brain monitoring devices. While the spike signal data described above is related to neural data, the spike sorting methods and systems described herein are not limited neural data. In general, data generated from any phenomenon, where the data can be represented as signals and where one or more signal characteristics can be used for classification of the signals, can be analyzed using the systems and methods described herein. As such, data relating to online search, online advertising, and/or electronic commerce can be analyzed using various embodiments for data mining, marketing, machine learning, bioinformatics, image analysis, etc.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A computer implemented method for spike sorting, the method comprising:
(a) receiving, in memory, data representing a plurality of spike signals;
(b) identifying a set of dimensions associated with the plurality of spike signals, each dimension in the set corresponding to a feature of the spike signals;
(c) selecting a first subset of dimensions from the set of dimensions;
(d) for each dimension in the first subset, computing by a processor a corresponding dimensional importance;
(e) for each dimension in the first subset, scaling by the processor the corresponding feature in the spike signals according to the corresponding dimensional importance; and
(f) clustering the plurality of spike signals having the scaled features to obtain an initial set of clusters.

2. The method of claim 1, wherein the set of dimensions comprises at least one of: a peak voltage corresponding to a channel for the spike signals, a principal component (PC) of a channel for the spike signals, and a peak PC of a channel for the spike signals.

3. The method of claim 1, wherein computing the corresponding dimensional importance for a dimension comprises:
projecting, by the processor, the spike signals along the dimension;
identifying, by the processor via iterative fuzzy c-means clustering of the projected spike signals, a clustering that maximizes a clustering score; and
designating, by the processor, a dimensional importance to the dimension based on the maximized clustering score.

4. The method of claim 3, wherein designating the dimensional importance comprises:
setting the dimensional importance to zero if the maximized clustering score is less than a selected threshold; and
otherwise setting the dimensional importance to a metric that results from a function of the number of clusters in the clustering that maximizes the clustering score.

5. The method of claim 4, wherein:
the selected threshold is 0.75; and
the metric is the square of a value that is the number of clusters minus one.

6. The method of claim 3, further comprising:
identifying a first dimension and a second dimension, wherein a first clustering corresponding to the first dimension is similar to a second clustering corresponding to the second dimension, the similarity being determined according to a similarity metric;
comparing the respective dimensional importances of the first and second dimensions; and
resetting the dimensional importance of the dimension having a lower dimensional importance to zero.

7. The method of claim 1, wherein clustering the plurality of spike signals having the scaled features comprises iterative fuzzy c-means clustering.

8. The method of claim 1, further comprising:
(g) for each cluster in the initial set of clusters:
selecting a second subset of dimensions from the set of dimensions, at least one dimension in the second subset being different from any dimension in the first subset;
for each dimension in the second subset, computing by the processor a corresponding dimensional importance;
for each dimension in the second subset, scaling by the processor the corresponding feature in the spike signals in the cluster according to the corresponding dimensional importance; and
clustering the spike signals having the scaled features in the cluster to obtain a subset of clusters within the cluster; and
(h) designating all subsets of clusters resulting from step (g) as a final set of clusters.

9. The method of claim 8, further comprising iteratively refining each cluster in the final set of clusters.

10. The method of claim 9, wherein iteratively refining a cluster comprises:
determining a cluster core; and
scaling each dimension in a plurality of dimensions associated with the cluster according to a distance, in the respective dimension, between the cluster core and spike signals in the cluster that are not within the cluster core.

11. The method of claim 9, further comprising filtering out a portion of the received data representing the plurality of spike signals using a first signal-to-noise ratio (SNR) threshold.

12. The method of claim 11, further comprising:
re-filtering the filtered out portion of the received data using a second SNR threshold that is lower than the first SNR threshold; and
repeating steps (b) through (h) using the re-filtered data representing the plurality of spike signals.

13. The method of claim 11, wherein the filtering comprises:
computing an SNR for each spike signal in the plurality of spike signals;
removing, from the data representing the plurality of spike signals, data corresponding to spike signals having an SNR less than the first SNR threshold.

14. The method of claim 11, wherein the filtering comprises:
spatially partitioning the spike signals into a plurality of bins;
computing a respective bin density for each bin; and
removing at least one spike signal from the data representing the plurality of spike signals, each removed spike signal having been partitioned into a bin having a density less than a threshold value, the threshold value resulting from a function of bin densities of neighboring bins.

15. The method of claim 9, further comprising:
prior to step (b), removing, from the data representing the plurality of spike signals, all but one spike signals that are near-simultaneous; and
after iteratively refining each cluster, adding each of the removed spike signals to a cluster according to a selected dimension of the respective removed spike signal.

16. The method of claim 9, further comprising:
computing at least one quality metric for each cluster in the final set of clusters; and
designating to each cluster a qualitative value, selected from the group consisting of good, medium, and bad, based on at least one corresponding quality metric.

17. The method of claim 16, wherein computing the at least one quality metric comprises computing at least one of: an Lratio for the cluster, a tightness of the cluster, an incompleteness of the cluster, a percentage of inter-spike intervals that are shorter than a specified threshold, and an isolation distance.

18. The method of claim 1, wherein the spike signals are obtained from a plurality of channels, the method further comprising:
aligning spike signals from each of the plurality of channels by aligning spike signal peaks across the plurality of channels.

19. The method of claim 1, further comprising:
removing spike signals in a selected time period if a number of spike signals in the selected time period exceeds a specified threshold.

20. The method of claim 1, further comprising:
determining, for at least one dimension of the clusters, a separability of each cluster in that dimension; and
dissolving a cluster that is determined to be separable.

21. A system comprising:
a first processor; and
a first memory coupled to the first processor, the first memory comprising instructions which, when executed by a processing unit comprising at least one of the first processor and a second processor, program the processing unit to:
(a) receive, in a memory module comprising at least one of the first memory and a second memory, data representing a plurality of spike signals;
(b) identify a set of dimensions associated with the plurality of spike signals, each dimension in the set corresponding to a feature of the spike signals;
(c) select a first subset of dimensions from the set of dimensions;
(d) for each dimension in the first subset, compute a corresponding dimensional importance;
(e) for each dimension in the first subset, scale the corresponding feature in the spike signals according to the corresponding dimensional importance; and
(f) cluster the plurality of spike signals having the scaled features to obtain an initial set of clusters.

22. An article of manufacture comprising a non-transitory storage medium having stored therein instructions which, when executed by a processor, program the processor to:
(a) receive, in memory coupled to the processor, data representing a plurality of spike signals;
(b) identify a set of dimensions associated with the plurality of spike signals, each dimension in the set corresponding to a feature of the spike signals;
(c) select a first subset of dimensions from the set of dimensions;
(d) for each dimension in the first subset, compute a corresponding dimensional importance;
(e) for each dimension in the first subset, scale the corresponding feature in the spike signals according to the corresponding dimensional importance; and
(f) cluster the plurality of spike signals having the scaled features to obtain an initial set of clusters.
